(19) United States
(12) Patent Application Publication
(10) Pub. No.: US 2006/0116576 A1
(43) Pub. Date: Jun. 1, 2006

(54) System and use thereof to provide indication of proximity between catheter and location of interest in 3-D space

(75) Inventors: David L. McGee, Sunnyvale, CA (US); N. Parker Willis, Atherton, CA (US)

Correspondence Address: Bingham McCuthen, LLP;Suite 1800, Three Embarcadero, San Franciso, CA, 94111-4067, US

(73) Assignee: Scimed Life Systems, Inc., Maple Grove, MN (US)

(21) Appl. No.: 11/002,629

(22) Filed: Dec. 1, 2004

(51) Int. Cl. A61M 25/00

(52) U.S. Cl. 600/434

(57) ABSTRACT

The present invention provides systems and method for navigating a medical probe (such as a catheter) relative to an anatomical body (such as a heart). A mark (such as a point or line), representing an anatomical region of interest (such as tissue targeted for treatment or tissue not targeted for treatment) is displayed on a representation of the anatomical body. The positions of the medical probe and the mark are determined within a three-dimensional coordinate system, and the proximity between the medical probe and the mark determined based on these positions. This proximity can then be indicated to a user, e.g., using graphics, text, or audible sounds.

### FIELD OF THE INVENTION

The present inventions generally relate to medical probes, and more particularly to systems and methods for navigating medical probes within anatomical organs or other anatomical structures.

### BACKGROUND OF THE INVENTION

It is often necessary or desirable to determine the location of a medical probe relative to a location of interest within three-dimensional space. In many procedures, such as interventional cardiac electrophysiology therapy, it is important for the physician to know the location of a probe, such as a catheter, (especially, a therapeutic catheter) relative to the patient's internal anatomy. During these procedures, a physician, e.g., steers an electrophysiology mapping catheter through a main vein or artery into the interior region of the heart that is to be treated. The physician then determines the source of the cardiac rhythm disturbance (i.e., the targeted cardiac tissue) either strictly by anatomical considerations or by placing mapping elements carried by the catheter into contact with the heart tissue, and operating the mapping catheter to generate an electrophysiology map of the interior region of the heart. Having identified the targeted cardiac tissue, the physician then steers an ablation catheter (which may or may not be the same catheter as the mapping catheter above) into the heart and places an ablating element carried by the catheter tip near the targeted cardiac tissue, and directs energy from the ablating element to ablate the tissue and form a lesion, thereby treating the cardiac disturbance.

In certain advanced electrophysiology procedures, it is desirable to create a linear lesion (or encircling lesion). For example, as part of the treatment for certain categories of atrial fibrillation, it may be desirable to create a curvilinear lesion around the pulmonary veins (PVs) and a linear lesion connecting one or more of the PVs to the mitral valve annulus. To do this, a physician must be able to move the ablation catheter tip along a desired path and either deliver ablative energy while slowly dragging the tip along the path, or deliver energy at a number of discrete points along that path. Either way, it is crucial that the physician define the desired path in three-dimensional space and be able to accurately and controllably move the catheter tip along that path. More importantly, during the electrophysiology procedure, it is important to prevent inadvertent damage to certain non-targeted regions, such as the atrioventricular (AV) and sinoatrial (SA) nodes, which control the natural electrical rhythm of the heart. Similarly, if the physician desires to electrically isolate PVs using ablation, it is important to prevent inadvertent damage to the pulmonary veins themselves, which could produce stenosis of the pulmonary veins.

Traditionally, navigation of catheters relative to points of interest has been accomplished using fluoroscopy. In this case, radiopaque elements are located on the distal end of the catheter and fluoroscopically imaged as the catheter is routed through the body. As a result, a two-dimensional image of the catheter, as represented by the illuminated radiopaque elements, is generated, thereby allowing the physician to roughly determine the location of the catheter. The use of fluoroscopy in locating catheters is somewhat limited, however, in that the physician is only able to visualize the catheter in two dimensions. In addition, fluoroscopy does not image soft tissues, making it difficult for the physician to visualize features of the anatomy as a reference for the navigation. Thus, fluoroscopy is sub-optimal for the purpose of navigating a catheter relative to anatomical structure composed primarily of soft tissues, e.g., within the heart.

Various types of technologies have been developed, or at least conceived, to address this issue. Recent advancements in transducer and processing technology have enabled commercially available real-time three-dimensional acoustic imaging of the heart and surrounding vasculature. For example, the SONOS 7500 imaging system, marketed by Philips Medical System located in Bothell, Wash., is an example of one such commercially available system that uses an external device to generate the image. This system provides real-time three-dimensional images of cardiac structures with resolution that, in some situations, may be adequate for assisting in catheter navigation and placement during electrophysiology procedures. See, e.g., Lang et al., “A Fantastic Journey: 3D Cardiac Acoustic Goes Live,” Radiology Management, November/December 2002; and “Phillips Prepares to Launch System Upgrade Capable of True Real-Time 3D Echo,” Diagnostic Imaging Scan, The Global Biweekly of Medical Imaging, Vol. 16, No. 18, Sep. 11, 2002, the disclosures of which are hereby expressly incorporated herein by reference.

U.S. Pat. Nos. 6,353,751 and 6,490,474 describe a system that can be used to navigate a catheter relative to previously recorded signals and ablation locations. The system includes a basket assembly of mapping electrodes that can be deployed within a chamber of a heart. Once deployed, the basket electrodes can be used to map the heart in order to identify and locate the tissue region to be therapeutically treated, e.g., by identifying the specific basket electrode that is adjacent the tissue region. An ablation catheter can then be introduced into the heart chamber and navigated relative to the basket by wirelessly transmitting electrical signals between the electrodes on the basket assembly and a positioning electrode located on the distal end of a catheter. An ablation electrode on the catheter, which may be the same as the positioning electrode, can then be navigated relative to the basket electrodes, and thus, placed adjacent the target tissue region and operated to create a lesion.

In other catheter navigation systems, a graphical representation of the catheter or a portion thereof is displayed in a three-dimensional computer-generated representation of a body tissue, e.g., a heart chamber. The three-dimensional representation of the body tissue is produced by mapping the geometry of the inner surface of the body tissue in a three-dimensional coordinate system, e.g., by moving a mapping device to multiple points on the body tissue. The position of the device to be guided within the body tissue is determined by placing one or more location elements on the device and tracking the position of these elements within the three-dimensional coordinate system. An example of this type of guidance system is the Realtime Position Management™ (RPM) tracking system, developed commercially by Boston Scientific Corporation and described in U.S. Pat. No. 6,216,027 and U.S. patent application Ser. No. 09/128,304, entitled “A Dynamically Alterable Three-Dimensional Graphical Model of a Body Region,” and the CARTO EP Navigation System, developed commercially by Biosense Webster and described in U.S. Pat. No. 5,391,199.

Although the previously described three-dimensional navigation systems have been particularly useful in generally displaying at least a portion of the catheter relative to its three-dimensional surroundings, it is still difficult for the physician to ascertain the proximity between the catheter tip and an anatomical region of interest. This is mainly due to the fact that the three-dimensional graphical image of the organ, e.g., the heart, is projected onto a two-dimensional screen, thereby providing a lack of depth perception. That is, the physician may only perceive two dimensions (length and width) at any given time. This problem can be better understood with reference to FIGS. 1 and 2. In FIG. 1, a three-dimensional graphical image of a heart 10 in which there is introduced a catheter 12 is shown on a computer screen 14. A mark, and in particular a line marking 16, representing a targeted ablation line, is shown graphically drawn on the heart wall. From the physician's point of view, the tip 18 of the catheter 12 appears to be in close proximity to the line marking 16 located on the heart wall. However, as shown in FIG. 2 (which represents a different viewing angle of the three-dimensional graphical image of the heart 10), the catheter tip 18 is located a relatively great distance from the line marking 16, and thus, the catheter tip 18 is not actually in close proximity to the targeted ablation line. Although it is possible for the physician to rotate the heart image to perceive all three-dimensions of the catheter tip 18 relative to the line marking 16 (e.g., by rotating between the heart images illustrated in FIGS. 1 and 2), the physician must continuously do this as the catheter tip 18 is moved. That is, the physician must rotate and view the image, then move the catheter tip, then rotate and view the image, etc. As a result, navigation of a catheter 12 relative to an anatomical region of interest within a three-dimensional environment may be tedious and time consuming. Furthermore, the perceived distance between two objects may be greatly influenced by the scale at which the objects are displayed, thereby possibly introducing errors in catheter navigation.

There thus remains a need for an improved system and method for navigating a catheter within a three-dimensional environment relative to an anatomical region of interest.

### SUMMARY OF THE INVENTION

In accordance with a first aspect of the present inventions, a method of navigating a medical probe (e.g., a catheter) to an anatomical body (such as a heart) is provided. Although the medical probe may be any probe that can perform a diagnostic or therapeutic procedure on the anatomical body, the present invention lends itself particularly well to the navigation of therapeutic medical probes, such as tissue ablative probes, relative to anatomical bodies that require precise targeted therapy.

The method comprises displaying a representation of the anatomical body, and optionally the medical probe, within a three-dimensional coordinate system. In one method, the representation(s) is graphically generated, but can also be generated using other means, such as Magnetic Resonance Imaging (MRI) or computed tomography (CT). The method further comprises displaying a mark (e.g., a point or a line) representing the location of an anatomical region of interest within the coordinate system. For example, the anatomical region of interest may be tissue targeted for treatment (e.g., cardiac tissue surrounding a pulmonary vein) or tissue not targeted for treatment (the atrioventricular (AV) and sinoatrial (SA) nodes). In one method, the mark is generated using a pointing device (e.g., a mouse and associated cursor).

The method further comprises determining positions of the medical probe and the mark within the coordinate system, and indicating the proximity between the medical probe and mark in real-time based on the determined positions. The proximity between the medical probe and the mark can be indicated in any one of a variety of manner. For example, the proximity can be indicated visually, e.g., by using text or graphics. Or, the proximity can be indicated audibly, e.g., by using beeps. The proximity indication may be binary (i.e., an indication of whether the medical probe is either “adjacent to” or “not adjacent to” the mark) or progressive (i.e., a continuous or discrete indication of different distances as the distance between the medical probe and the mark varies).

In accordance with a second aspect of the present inventions, a medical navigation system for navigating the previously described medical probe relative to an anatomical body (such as a heart) is provided. The navigation system comprises a pointing device (such as a mouse) that allows a user to specify the location of a mark or marks (e.g., a point or line) on an image of the anatomical body. The navigation system may optionally comprise a graphical processor for generating the representation of the anatomical body. Alternatively, the navigation system may comprise other imaging means, such as an MRI or CT scanner.

The navigation system further comprises one or more processors configured for determining positions of the medical probe and the user specified mark within a three-dimensional coordinate system. If a graphical processor is provided, it preferably is also configured to generate representations of the medical probe and mark based on the determined operative probe and mark positions. In one embodiment, the navigation system comprises one or more location elements disposed on the medical probe, in which case, the processor(s) may comprise a localization processor configured for determining the location element position(s) within the coordinate system. The position of the probe can then be derived from the determined location element position(s).

The processor(s) are also configured for determining a proximity between the medical probe and mark based on the determined probe and mark positions. The navigation system further comprises an output device (such as a monitor or speaker) configured to indicate the proximity between the medical probe and the mark to the user. The proximity between the medical probe and the mark can be indicated in any one of the previously described manners.

Other objects and features of the present invention will become apparent from consideration of the following description taken in conjunction with the accompanying drawings.

### BRIEF DESCRIPTION OF THE DRAWINGS

The drawings illustrate the design and utility of preferred embodiments of the present invention, in which similar elements are referred to by common reference numerals. In order to better appreciate how the above-recited and other advantages and objects of the present inventions are obtained, a more particular description of the present inventions briefly described above will be rendered by reference to specific embodiments thereof, which are illustrated in the accompanying drawings. Understanding that these drawings depict only typical embodiments of the invention and are not therefore to be considered limiting of its scope, the invention will be described and explained with additional specificity and detail through the use of the accompanying drawings in which:

FIG. 1 is a front view of a display illustrating an image of a catheter within a heart;

FIG. 2 is a view of the objects displayed in FIG. 1, but from a rotated viewing angle;

FIG. 3 is a functional block diagram of one preferred embodiment of a catheter navigation system constructed in accordance with the present inventions;

FIG. 4 is a plan view of a mapping/ablation catheter used in the navigation system of FIG. 3;

FIG. 5 is a plan view of a reference catheter used in the navigation system of FIG. 3; and

FIG. 6 is a front view of a monitor displaying the mapping/ablation and reference catheters illustrated in FIGS. 4 and 5 within a heart marked for ablation.

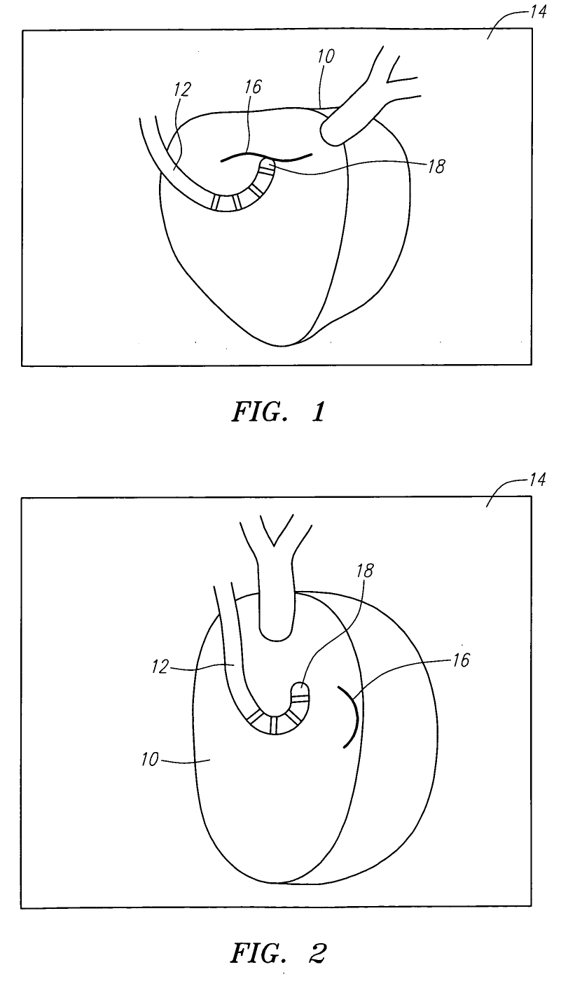

### DETAILED DESCRIPTION OF THE PREFERRED EMBODIMENTS

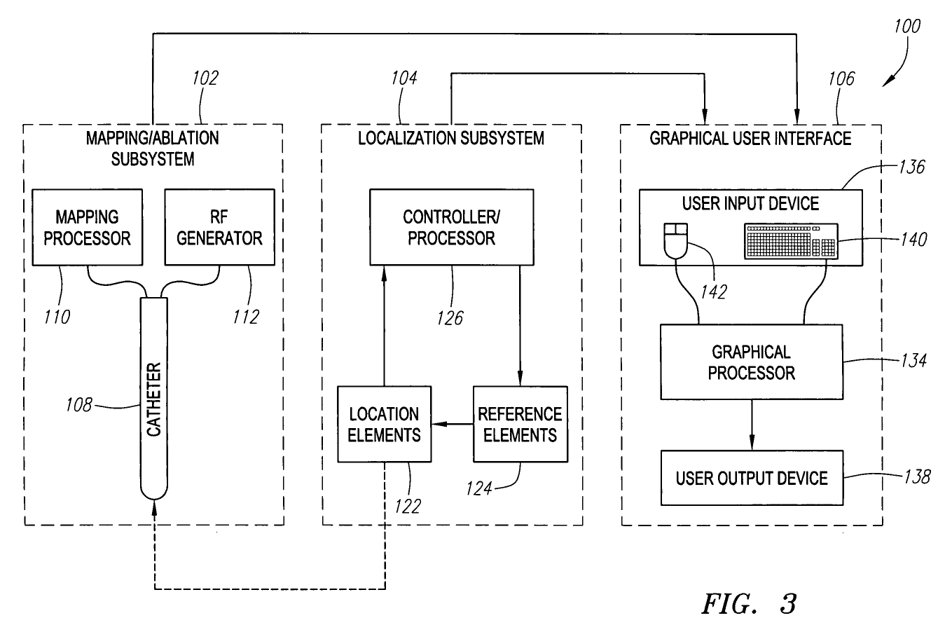

Referring to FIG. 3, an exemplary catheter navigation system 100 constructed in accordance with the present invention is shown. The navigation system 100 is particularly suited for mapping and treating the heart with catheters. Nevertheless, it should be appreciated that it can be used for treating other internal anatomical structures, e.g., the prostrate, brain, gall bladder, uterus, esophagus and other regions in the body, and can be used to navigate medical devices other than catheters.

The navigation system 100 generally comprises (1) a mapping/ablation subsystem 102 for mapping and ablating tissue within the heart; (2) a localization subsystem 104 for registering mapping data and the movement of a probe within a three-dimensional coordinate system; and (3) a graphical user interface 106 configured for generating and displaying graphics of the heart, mapping data, and probe within the three-dimensional coordinate system. The graphical user interface 106 is also configured for generating and displaying user-defining markings of anatomical regions of interest within the three-dimensional coordinate system, as well as providing an indication of the proximity between the probe and such markings.

It should be noted that the elements illustrated in FIG. 3 are functional in nature, and are not meant to limit the structure that performs these functions in any manner. For example, several of the functional blocks can be embodied in a single device, or one of the functional blocks can be embodied in multiple devices. Also, the functions can be performed in hardware, software, or firmware.

I. Mapping/Ablation Subsystem

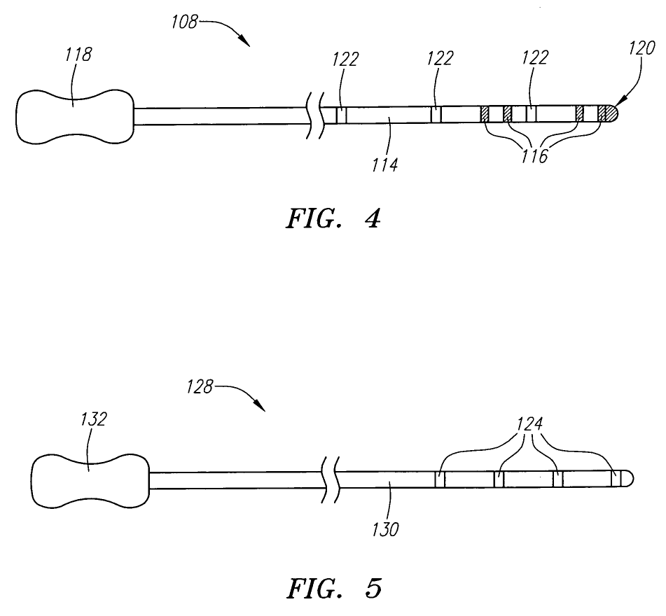

The mapping/ablation subsystem 102 is configured to identify and treat a target tissue site or sites, e.g., aberrant conductive pathways. To this end, the mapping/ablation subsystem 102 comprises a mapping/ablation catheter 108, a mapping processor 110, and a radio frequency (RF) generator 112. As further illustrated in FIG. 4, the mapping/ablation catheter 108 comprises an elongate catheter member 114, a plurality of electrodes 116 (in this case, four) carried at the distal end of the catheter member 114, and a handle 118 carried at the proximal end of the elongate member 114. All four electrodes 116 on the catheter member 114 are configured to detect electrical signals in the myocardial tissue for subsequent identification of target sites. The electrode 116 at the distal tip 120 of the catheter member 114 is also configured to be used as an ablation electrode to provide ablation energy to the targeted sites when placed adjacent thereto and operated. The handle 118 includes an electrical connector (not shown) for electrical coupling to the mapping processor 110 and RF generator 112.

Referring back to FIG. 3, the mapping processor 110 is configured to derive activation times and voltage distribution from the electrical signals obtained from the electrodes 116 to determine irregular electrical signals within the heart, which can then be graphically displayed as a map. Mapping of tissue within the heart is well known in the art, and thus for purposes of brevity, the mapping processor 110 will not be described in further detail. Further details regarding electrophysiology mapping are provided in U.S. Pat. Nos. 5,485,849, 5,494,042, 5,833,621, and 6,101,409, which are expressly incorporated herein by reference.

The RF generator 112 is configured to deliver ablation energy to the ablation electrode (i.e., the distal most electrode 116) in a controlled manner in order to ablate sites identified by the mapping processor 110. Alternatively, other types of ablative sources besides the RF generator 112 can be used, e.g., a microwave generator, an acoustic generator, a cryoablation generator, and a laser or other optical generator. Ablation of tissue within the heart is well known in the art, and thus for purposes of brevity, the RF generator 112 will not be described in further detail. Further details regarding RF generators are provided in U.S. Pat. No. 5,383,874, which is expressly incorporated herein by reference.

It should be noted that other types of mapping/ablation catheters can be used in the navigation system 100. For example, a catheter having a basket structure of resilient splines, each of which carries a plurality of dedicated mapping electrodes can be used. This catheter may be placed in a heart chamber, so that the resilient splines conform to the endocardial surface of the heart, thereby placing and distributing the mapping electrodes along the entire endocardial surface of the cavity for efficient mapping. The catheter may also have a roving ablation electrode that can be steered in contact with the ablation sites identified by the mapping electrodes. Or a separate ablation catheter with a dedicated ablation electrode or electrodes can be used.

II. Localization Subsystem

The localization subsystem 104 includes a plurality of location elements 122, a plurality of reference elements 124, and a controller/processor 126 coupled to the reference elements 124 and location elements 122. As shown in FIG. 4, the location elements 122 (in this case, three) are carried by the distal end of the mapping/ablation catheter 108. As shown in FIG. 5, at least some of the reference elements 124 are carried by a reference catheter 128. Like the mapping/ablation catheter, the reference catheter 128 comprises an elongate catheter member 130 and a handle 132 carried at the proximal end of the elongate member 130. The distal end of the reference catheter 128 may optionally comprise a plurality of electrodes (not shown), e.g., to provide the reference catheter 128 with mapping functionality. The reference catheter 128 may be affixed within selected regions of the heart in order to establish an internal three-dimensional coordinate system, as will be further discussed below. Alternatively, the reference elements 124 may be located outside of the patient's body, e.g., affixed to the patient's skin, in order to establish an external three-dimensional coordinate system.

In any event, the controller/processor 126 can establish a three-dimensional coordinate system by controlling and processing signals transmitted between the spaced apart reference elements 124. In essence, the three-dimensional coordinate system provides an absolute framework in which all spatial measurements will be taken. The controller/processor 126 can also determine the positional coordinates of the location elements 122, and thus the distal end of the mapping/ablation catheter 108, within this coordinate system. As will be described in further detail below, this positional information can ultimately be used to graphically reconstruct the heart or heart chamber and the distal end of the mapping/ablation catheter 108 (as well as any reference catheters 128), track the movement of the mapping/ablation catheter 108 within the heart chamber, and, in conjunction with the mapping data obtained from the mapping processor 110, generate an electrophysiological map.

In the illustrated embodiment, the localization subsystem 104 employs ultrasound triangulation principles to determine the coordinates of the location elements 122 carried by the mapping/ablation catheter 108. In this case, the location and reference elements 122, 124 take the form of ultrasound transducers. The coordinates of the location elements 122 can be determined within an internal reference frame established by arranging the reference elements 124 in three-dimensional space. For example, the first two dimensions of the coordinate system can be provided by placing a reference catheter 128 within the coronary sinus (CS), thereby disposing its reference elements 124 in a two-dimensional plane. The third dimension can be provided by placing another reference catheter 128 within the right ventricular (RV) apex to dispose its reference elements 124 off of the two-dimensional plane. Notably, only four reference elements 124 are needed to provide the three dimensions. Any remaining reference elements 124 can be used to improve the accuracy of the triangulation process.

The controller/processor 126 is operated to sequentially transmit ultrasound pulses (e.g., 500 KHz pulses) through each reference element 124, and then measure the time delay between the respective transmit and receive pulses at the location element 122 and other reference elements 124. The controller/processor 126 then calculates the relative distances between each reference element 124 and the remaining reference elements 124 and location elements 122 using the “time of flight” and velocity of the ultrasound pulses. The distance information can be calculated as d=vt, where d is the distance between the transmitter and receiver, v is the velocity of the ultrasound signal within the medium (i.e., blood), and t is the time delay. To simplify the distance computations, the velocity of the ultrasound pulses may be assumed to be constant. This assumption typically only produces a small error when the reference elements 124 are located inside the body, since the velocity of ultrasound propagation is approximately the same in body tissue and blood.

The controller/processor 126 then establishes a three-dimensional coordinate system by triangulating the distances between the reference elements 124, and determines the positions of each of the location elements 122 within that coordinate system by triangulating the distances between the reference elements 124 and the location elements 122. Additional details on determining the positions of ultrasound transducers within a three-dimensional coordinate system can be found in U.S. Pat. No. 6,490,474 and U.S. patent application Ser. No. 09/128,304, entitled “A dynamically alterable three-dimensional graphical model of a body region,” which are fully and expressly incorporated herein by reference.

It should be noted that there are other means for determining the positions of catheters within a three-dimensional coordinate system. For example, magnetic tracking techniques, such as that disclosed in U.S. Pat. No. 5,391,199, which is expressly incorporated herein by reference, can be employed. As another example, a voltage tracking technique, such as that disclosed in U.S. Pat. No. 5,983,126, which is expressly incorporated herein by reference, can be employed.

III. Graphical User Interface

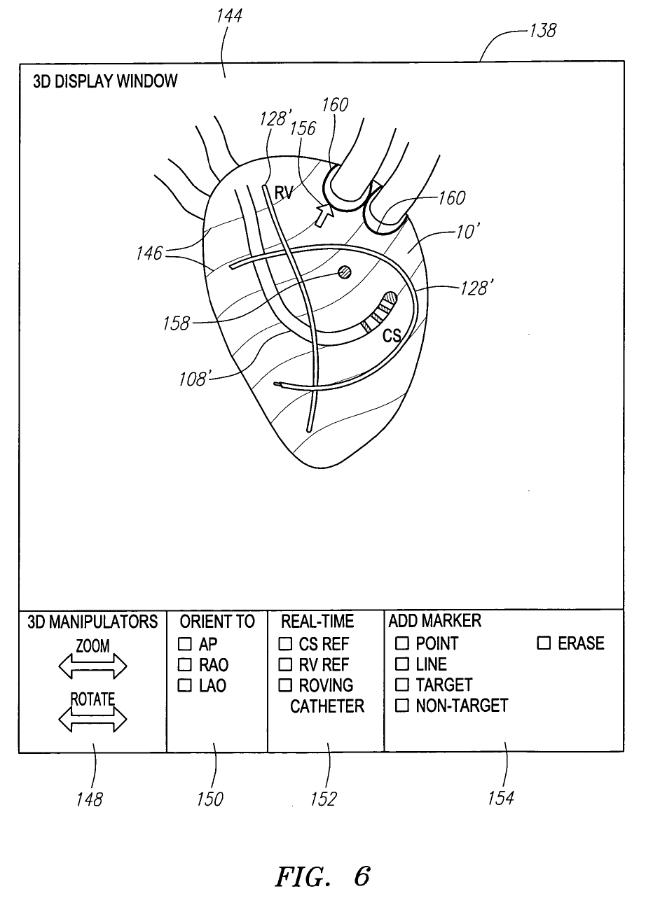

The graphical user interface 106 comprises a graphical processor 134, a user input device 136, and an output device 138 (and specifically, a monitor). The graphical processor 134 is configured for generating a representation of an internal anatomical structure (in this case, the heart) in the form of a computer-generated reconstruction 10′ within the coordinate system, which is then displayed in a 3-D display window 144 on the monitor 138, as illustrated in FIG. 6. The three-dimensional graphical processor 134 accomplishes this by acquiring the positions of the location elements 122 within the coordinate system from the localization subsystem 104 as the mapping/ablation catheter 108 is moved around within the cavity of the internal anatomical structure, and then deforming a graphical anatomical shell to the acquired positions.

Instead of, or in addition to, graphically reconstructing the body tissue, any one of a number of imaging techniques may be used to generate a three-dimensional image of the body tissue. For example, a Magnetic Resonance Imaging (MRI) imager, or a Computed Tomography (CT) imager can be used to generate a three-dimensional image of the internal anatomical structure. To accomplish this, the imager may be moved laterally and/or rotationally to obtain multiple cross-sectional or sector images of the body tissue at different positions within the body tissue. The multiple cross-sectional images may then be aggregated (i.e., pieced together) to reconstruct a three-dimensional image of the internal anatomical structure. The three-dimensional image of the internal anatomical structure may be registered within the coordinate system by tracking the position of the imager, and therefore the cross-sectional or sector images taken by the imager, for example, by attaching location elements to the imager. Alternatively, the position of anatomic landmarks within the body tissue may be determined in the coordinate system, e.g., using the mapping/ablation catheter 108 or a pointing device, such as a mouse. The three-dimensional image of the internal anatomical structure may then be scaled and registered with the coordinate system by correlating the positions of the anatomic landmarks in the three-dimensional image of the internal anatomical structure with the determined positions of the anatomic landmarks in the coordinate system.

The graphical processor 134 is also configured for generating a graphical representation 108′ of the mapping/ablation catheter 108 within the established three-dimensional coordinate system, which is then superimposed over the graphical heart representation 10′ in the 3D display window 144, as illustrated in FIG. 6. The graphical processor 134 can generate the graphical catheter representation 108′ from a pre-stored graphical model of the catheter 108, which can be deformed in accordance with the calculated positional coordinates of the location elements 122 carried by the catheter 108. In the illustrated embodiment, the graphical catheter representation 108′ is dynamically generated in real-time. That is, the catheter representation 108′ is graphically generated in successive time periods (e.g., once every heartbeat), so that it moves and bends as the actual catheter 108 is moved and bent within the heart chamber. The graphical processor 134 may optionally be configured to generate graphical representations 128′ of the reference catheters 128 in real-time, as illustrated in FIG. 6.

The graphical processor 134 is also configured for generating an electrical activity map 146 within the three-dimensional coordinate system, which is then superimposed over the graphical heart representation 10′ in the 3D display window 144, as illustrated in FIG. 6. The graphical processor 134 can generate the electrical activity map 146 based on the electrical activity information acquired from the mapping/ablation subsystem 102 and the positions of the mapping electrodes 116 geometrically derived from the positions of the location elements 122 obtained from the localization subsystem 104. This electrical activity map illustrates sites of interest, e.g., electrophysiology recording and ablation sites, for providing subsequent ablative treatment, and can be provided in the form of an isochronal or isopotential map. The electrical activity information may also be displayed separately from the 3D display window 144.

Additional details on graphically generating anatomical structures, catheters, and electrical activity maps within a three-dimensional environment can be found in U.S. Pat. No. 6,490,474 and U.S. patent application Ser. No. 09/128,304, entitled “A dynamically alterable three-dimensional graphical mode of a body region,” which have previously been incorporated herein by reference.

The user input device 136 allows the user to interact with the graphics displayed on the monitor 138, and comprises a standard keyboard 140 and a graphical pointing device 142, such as a mouse. The graphical processor 134 responds to the user input device 136 by manipulating the graphics within the 3D display window 144. As an example, the user may rotate the 3D display window 144 in three-dimensions and “zoom” towards or away from the window 144 by clicking on the appropriate icon in the manipulation box 148 using the mouse 142. The user may also select one of the standard orientations, used in fluoroscopy, such as anterior-posterior (AP), lateral, right anterior oblique (RAO) or left anterior oblique (LAO) by selecting the appropriate icon in orientation box 150 using the mouse 142. The user may also select which catheters to display in real-time by checking the appropriate icons in the real-time box 152 using the mouse 142.

Using the mouse 142, the user can also mark anatomical regions of interest on the heart model by placing a cursor 156 at the appropriate location on the graphical heart representation 10′ and clicking. In the illustrated embodiment, the user can either mark the graphical heart representation with point markings 158 or with line markings 160 (either linear or curvilinear). For example, if the user desires to place a point marking 158 at an anatomical region of interest, the appropriate icon in the marking box 154 can be clicked, and then the user can mark the graphical heart representation 10′ by moving the cursor 156 to a selected region on the graphical heart representation 10′ and clicking the mouse 142. The graphical heart representation 10′ can be marked with additional points markings 158 in the same manner. If the user desires to place a line marking 160 at an anatomical region of interest, the appropriate icon in the marking box 154 can be clicked, and then the user can mark the graphical heart representation 10′ by clicking the mouse 142, and dragging the cursor 156. If curvilinear, the line marking 160 may either be open or closed. The user may also erase marks 158/160 from the graphical heart representation 10′ by clicking on the appropriate icon in the marking box 154, and them moving the cursor 156 over the mark 158/160, while clicking the mouse 142.

The user may also designate the marked anatomical regions as either tissue that is targeted for treatment (in this case, ablation) or tissue that is not targeted for treatment—typically tissue that should not be ablated. In particular, prior to marking the graphical heart representation 10′ as previously described, the user determines whether an anatomical region is targeted tissue or non-targeted tissue, and then clicks the appropriate icon in the marking box 154. Marks designating targeted tissue and marks designating non-targeted tissue can be distinguished from each other in order to remind the user during the ablation procedure which anatomical regions are to be ablated and which anatomical regions are not to be ablated. For example, marks designating targeted tissue can be generated and displayed with a particular color, such as green, to indicate that the corresponding anatomical regions are safe, and in fact, desirable, to ablate. Marks designating non-targeted tissue can be generated and displayed with another color, such as red, to indicate the corresponding anatomical regions are not safe to ablate.

As the marks are being made by the user, the graphical processor 134 transforms the x-y coordinate system of the cursor 156 into the established three-dimensional coordinate system using standard coordinate transformation techniques, so that the graphical processor 134 can superimpose the marks over the graphical heart representation 10′. Because the three-dimensional heart representation 10′ is projected onto the two-dimensional display window 144, the graphical processor 134 will superimpose the marks onto the front wall of the graphical heart representation 10′, as perceived by the user. If the user desires to place marks on the back wall or side wall of the graphical heart representation 10′, or if the user desires to extend the marks from the front wall around to the side wall or back wall of the graphical heart representation 10′, the graphical heart representation 10 need only be rotated using the rotation feature in the manipulation box 148, so that the previously perceived back wall or side wall of the graphical heart representation 10 currently becomes the front wall of the graphical heart representation 10′, as perceived by the user. Alternatively, the graphical processor 134 allows the user to graphically cutaway the front wall of the graphical heart representation 10′ to expose the back wall. In this case, the user may define marks on the back wall of the graphical heart representation 10′ through the cutout without having to rotate graphical heart representation 10′.

It should be noted that pointing devices other than a mouse and associated cursor can be used define marks on the graphical heart representation 10′. For example, the mapping/ablation catheter 108 or a marking catheter with location elements may alternatively be used to place marks on the graphical heart representation 10′. In this case, the graphical processor 134 need not perform a coordinate transformation, since the catheter 108 or marking catheter is already tracked within the three-dimensional coordinate system.

The graphical processor 134 is also configured to provide the user with an indication of the proximity between the tip 120 of the mapping/ablation catheter 108 and any marks that have been defined on the graphical heart representation 10′. In particular, the graphical processor 134 geometrically calculates, in real-time, the distance between the catheter tip 120, as deduced from the calculated positions of the location elements 122, and the marks, and in particular, the point marking 158 or the closest point in a line marking 160. The graphical processor 134 may provide an indication of this distance to the user in any one of a variety of manners. For example, the proximity indication can be visually conveyed to the user through the use of text or graphics, or audibly conveyed to the user through beeps or other sounds.

In the illustrated embodiment, the proximity indication is binary in that the graphical processor 134 only provides the user within an indication of when the catheter tip 120 is “close to” or “not close to” the mark. The threshold distance that dictates whether the proximity between the catheter tip 120 and the mark is close can exist in the form of a default value and/or can be defined or adjusted by the user. To provide the user with a binary proximity indication, the graphical processor 134 can, e.g., toggle the mark or other proximity-indicating graphical element between two colors, toggle a graphical symbol adjacent the mark or catheter on and off, or provide audible sounds.

The binary proximity indication technique works particularly well when the mark is a line marking 160 that designates target tissue. For example, as the user attempts to move the catheter tip 120 along a path defined by the line marking 160, the graphical processor 134 may display the line marking 160 or another graphical element with a green color to indicate that the catheter tip is “on-the-path,” and may display the line marking 160 or other graphical element with a red or black color to indicate that the catheter tip is “off-the-path.” Thus, the user will be provided with real-time feedback that facilitates guidance of the catheter tip 120 along the desired path designated by the line marking 160. This is particularly critical during a therapy procedure, which helps ensure that the linear ablation lesion is being created along the targeted tissue.

The binary proximity indication technique also works particularly well when the mark (whether in the form of a point marking 158 or line marking 160) designates non-targeted tissue, i.e., tissue the ablation of which should or must be avoided. For example, if the catheter tip 120 becomes dangerously close to a marking (as defined by the threshold distance) designating non-targeted tissue, the graphical processor 134 can generate a visual alarm (e.g., a flashing symbol) or an audible alarm (such as a series of beeps) that immediately warns the user not to ablate tissue in that region. Thus, the user will be provided with real-time feedback that helps ensure that the user does not inadvertently deliver therapy to site that should be avoided.

In an alternative embodiment, the proximity indication may be progressive in that the graphical processor 134 provides the user within an indication of one of many distances between the catheter tip 120 and the mark as the catheter tip 120 is moved. The graphical processor 134 can provide the progressive proximity indication in a discrete manner, e.g., by changing the mark or other proximity-indicating graphical element between various colors (e.g., green, blue, yellow, orange, and red indicate respective distances of 1, 2, 3, 4, and 5 mm), or a continuous manner, e.g., by displaying text indicating the actual real-time distance between the catheter tip 120 and the mark. In the case of progressive proximity indications that are discrete, the threshold distances can exist in the form of a default value and/or can be defined or adjusted by the user.

Having described the structure of the navigation system 100, one method of using the system 100 to locate and treat an aberrant conductive pathway within the heart 10, such as those typically associated with ventricular tachycardia or atrial fibrillation, will now be described. First, under fluoroscopy, the reference catheters 128 are intravenously introduced into the heart 10, and in particular, within the coronary sinus (CS) and right ventricle (RV) apex, so that the reference elements 124 are fixed within a three-dimensional arrangement. During introduction of the reference catheters 128, the localization subsystem 104 may be operated to transmit signals between the reference elements 124, so that the locations of the distal ends of the reference catheters 128 can be determined and graphically displayed in the 3D display window 144 on the monitor 138. Next, the mapping/ablation catheter 108 is introduced into the appropriate chamber of the heart 10 under fluoroscopy. For example, if the disease to be treated is ventricular tachycardia, the catheter 108 will be introduced into the left ventricle. If the disease to be treated is atrial fibrillation, the catheter 108 will be introduced into the left atrium. During this time period, the localization subsystem 104 may be operated to transmit signals between the reference elements 124 and the location elements 122, so that the locations of the distal end of the catheter 108 can be determined and graphically displayed in the 3D display window 144.

The catheter 108 is then moved around within the selected chamber of the heart 10 as the position of the distal tip 120 is determined. The graphical processor 134 generates the graphical heart representation 10′ by deforming the graphical model of the heart to coincide with the positions of the distal tip 120 as they are acquired. Once the graphical heart representation 10′ is created, the mapping processor 110 is then operated to record electrical activity within the heart 10 and derive mapping data therefrom. The graphical processor 134 acquires this mapping data and generates the electrical activity map 146, which is then displayed on the 3D display window 144 over the graphical heart representation 10′.

If an aberrant region is identified, the user will then use the mouse 142 to mark this region as targeted tissue. Using the mouse 142, the user may also mark the non-targeted tissue. The distal tip 120 of the mapping/ablation catheter 108 is then placed into contact with the targeted tissue mark, and the RF generator operated 112 to therapeutically create a lesion on the mark. If the targeted tissue mark is a point marking 158 or a series of point markings 158, the lesion will take the form of a spot lesion or lesions. If the targeted tissue mark is a line marking 160, the lesion will take the form of a linear or curvilinear lesion. During the ablation process, the graphical processor 134 will indicate the proximity of the catheter tip 120 relative to the targeted tissue mark, thereby ensuring that the user is therapeutically ablating the targeted tissue. Importantly, the graphical processor 134 will also indicate the proximity of the catheter tip 120 relative to the non-targeted tissue mark, thereby ensuring that the non-targeted tissue is not therapeutically ablated. After the ablation process is complete, the mapping processor 110 can again be operated to ensure that the heart disease has been successfully treated. If additional aberrant conductive pathways have been found, the marking and ablation steps can be repeated. If no aberrant conductive pathways have been found, the reference catheters 128 and mapping/ablation catheter 108 can then be removed from the patient.

Although particular embodiments of the present invention have been shown and described, it will be understood that it is not intended to limit the present invention to the preferred embodiments, and it will be obvious to those skilled in the art that various changes and modifications may be made without departing from the spirit and scope of the present invention. Thus, the present inventions are intended to cover alternatives, modifications, and equivalents, which may be included within the spirit and scope of the present invention as defined by the claims.

What is claimed is:

1. A method of navigating a medical probe relative to an anatomical body, comprising:
displaying a representation of the anatomical body within a three-dimensional coordinate system;
graphically displaying a mark representing the location of an anatomical region of interest within the coordinate system;
determining positions of the medical probe and the mark within the coordinate system; and
indicating the proximity between the medical probe and mark in real-time, based on the determined probe and mark positions.

2. The method of claim 1, wherein the representation of the anatomical body is graphical.

3. The method of claim 1, further comprising displaying a representation of the medical probe within the coordinate system.

4. The method of claim 1, wherein the medical probe is a therapeutic probe.

5. The method of claim 1, wherein the medical probe is an ablation probe.

6. The method of claim 1, wherein the medical probe is an intravascular catheter.

7. The method of claim 1, wherein the anatomical body is a heart.

8. The method of claim 1, further comprising generating the mark using a pointing device.

9. The method of claim 1, wherein the mark is a point.

10. The method of claim 1, wherein the mark is a line.

11. The method of claim 1, wherein the anatomical region of interest is tissue targeted for treatment.

12. The method of claim 1, wherein the anatomical region of interest is tissue that should be avoided during treatment.

13. The method of claim 1, wherein the proximity indication is visual.

14. The method of claim 13, wherein the visual indication is textual.

15. The method of claim 13, wherein the visual indication is graphical.

16. The method of claim 1, wherein the proximity indication is audible.

17. The method of claim 1, wherein the proximity indication is binary.

18. The method of claim 1, wherein the proximity indication is progressive.

19. A medical navigation system for navigating a medical probe relative to an anatomical body, comprising:
a pointing device that allows a user to specify the location of a mark on a representation of the anatomical body; and
one or more processors configured for determining positions of the medical probe and the user specified mark within a three-dimensional coordinate system, and for determining a proximity between the medical probe and mark based on the determined positions; and
an output device configured to indicate the proximity to the user.

20. The system of claim 19, further comprising one or more location elements disposed on the medical probe, wherein the one or more processors comprises a localization processor configured for determining positions of the one or more location elements within the coordinate system, wherein the probe position is derived from the one or more location element positions.

21. The system of claim 19, wherein the one or more processors comprises a graphics processor configured for generating the representation of the anatomical body.

22. The system of claim 19, wherein the one or more processors comprises a graphics processor configured for generating representations of the medical probe and the mark based on the respective determined probe and mark positions.

23. The system of claim 19, wherein the medical probe is a therapeutic probe.

24. The system of claim 19, wherein the medical probe is an ablation probe.

25. The system of claim 19, wherein the medical probe is an intravascular catheter.

26. The system of claim 19, wherein the anatomical body is a heart.

27. The system of claim 19, wherein the mark is a point.

28. The system of claim 19, wherein the mark is a line.

29. The system of claim 19, wherein the output device is a monitor.

30. The system of claim 29, wherein the proximity indication is textual.

31. The system of claim 29, wherein the proximity indication is graphical.

32. The system of claim 19, wherein the output device is a speaker

33. The system of claim 19, wherein the proximity indication is binary.

34. The system of claim 19, wherein the proximity indication is progressive.
